(19) United States
(12) Patent Application Publication
(10) Pub. No.: US 2026/0045361 A1
(43) Pub. Date: Feb. 12, 2026

(54) RAG-ENHANCED PROBLEM SOLVING FOR MEDICAL DECISION MAKING

(71) Applicant: NEC Laboratories America, Inc., Princeton, NJ (US)

(72) Inventors: Wei Cheng, Princeton Junction, NJ (US); Yanchi Liu, Princeton, NJ (US); LuAn Tang, Cranbury, NJ (US); Yiyou Sun, Madison, WI (US); Haifeng Chen, West Windsor, NJ (US); Ivan Xiong, New York, NY (US)

(21) Appl. No.: 19/296,145

(22) Filed: Aug. 11, 2025

Related U.S. Application Data
(60) Provisional application No. 63/681,934, filed on Aug. 12, 2024.

(51) Int. Cl. G16H 50/20; G06F 18/22; G06N 3/0475; G16H 10/60

(52) U.S. Cl. CPC G16H 50/20; G06F 18/22; G06N 3/0475; G16H 10/60

(57) ABSTRACT

Methods and systems include comparing a description of an issue to documents to generate similarity scores for the documents. A set of most-relevant documents are selected from the documents based on the similarity scores. A large language model (LLM) is prompted to generate a solution to the issue. A corrective action is performed based on the solution to correct the issue.

This application claims priority to U.S. Application No. 63/681,934 filed on Aug. 12, 2024, incorporated herein by reference in its entirety.

### BACKGROUND

### Technical Field

The present invention relates to retrieval augmented generation (RAG) and, more particularly, to correcting issues using augmented retrieval generation.

### Description of the Related Art

Complex systems are difficult to engineer in a manner that is free from bugs or other issues. Unexpected issues may arise, and it can be difficult to identify the root cause of the issue.

### SUMMARY

A method includes comparing a description of an issue to documents to generate similarity scores for the documents. A set of most-relevant documents are selected from the documents based on the similarity scores. A large language model (LLM) is prompted to generate a solution to the issue. A corrective action is performed based on the solution to correct the issue.

A system includes a hardware processor and a memory that stores a computer program. When executed by the hardware processor, the computer program causes the hardware processor to compare a description of an issue to a plurality of documents to generate similarity scores for the plurality of documents, to select a set of most-relevant documents from the plurality of documents based on the similarity scores, to prompt an LLM to generate a solution to the issue, and to perform a corrective action based on the solution to correct the issue.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram illustrating retrieval augmented generation (RAG)-based generation of a solution to an issue, in accordance with an embodiment of the present invention;

FIG. 2 is a block/flow diagram of a method of selecting a subset of documents relevant to an issue description, in accordance with an embodiment of the present invention;

FIG. 3 is a block/flow diagram of a method of solving an issue using RAG-based prompting to a large language model, in accordance with an embodiment of the present invention;

FIG. 4 is a block diagram illustrating RAG-based issue solving in a software engineering context, in accordance with an embodiment of the present invention;

FIG. 5 is a block diagram of a healthcare facility that uses RAG-based issue solving to diagnose and treat medical conditions, in accordance with an embodiment of the present invention;

FIG. 6 is a block diagram of a computing device that uses RAG-based issue solving to solve issues, in accordance with an embodiment of the present invention;

FIG. 7 is a diagram of an exemplary neural network architecture that can be used to implement part of an LLM, in accordance with an embodiment of the present invention; and

FIG. 8 is a diagram of an exemplary deep neural network architecture that can be used to implement part of an LLM, in accordance with an embodiment of the present invention.

### DETAILED DESCRIPTION OF PREFERRED EMBODIMENTS

Large language models (LLMs) can be used to help in engineering complex systems, such as when writing software or diagnosing and treating a patient's medical condition. When an issue arises, such as a bug in the software or a complication in the medical's health, the LLM can help to diagnose the issue and suggest solutions.

However, the most powerful LLMs are expensive to operate. These LLMs may accept a large number of input tokens, making it possible for the LLM to consider the full context of the issue when identifying the problem and determining the solution. In contrast, less powerful LLMs allow a smaller number of context tokens. While their reasoning capabilities may be similar to the more powerful LLMs, the limited scope of the context limits how much of the system can be described. This can result in the less powerful LLM missing the source of the issue or misidentifying the correct solution. However, executing prompts on the less powerful LLM may be significantly less computationally expensive than executing on the more powerful LLM.

Retrieval augmented generation can be used to rank the source information according to its relevance to the issue. In the context of software development, the source files for a software project may be vectorized to create vector representations and may then be compared to a textual description of the issue. In the context of medical treatment, the patient's medical records and background medical information may be similarly ranked. The most relevant source documents can be selected in this manner, limiting the size of the input information to the LLM. A less computationally expensive LLM can then be used while minimizing the risk that the source of the problem will be overlooked.

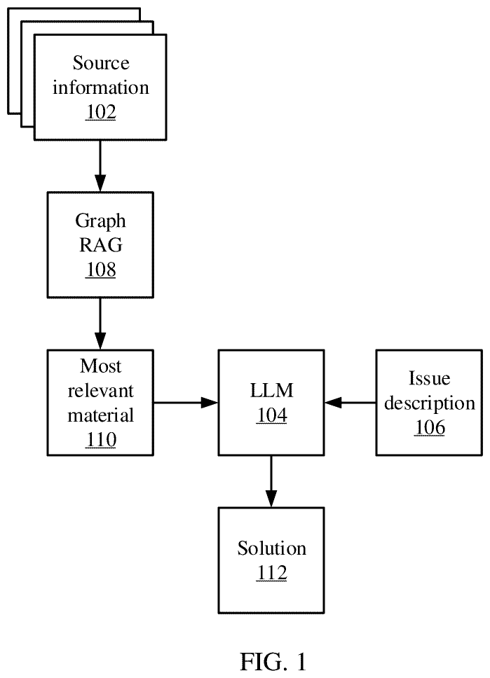

Referring now to FIG. 1, a system for correcting an issue is shown. A set of source information 102 is provided. In some embodiments the source information 102 may include source code for a software project and may furthermore include a full development environment. In some embodiments the source information 102 may include medical records for one or more patients, as well as a corpus of background medical information.

An issue description 106 is provided. The issue description 106 may include a natural language description of a software bug, for example identifying circumstances in which the software crashes or behaves unexpectedly, and may further include supporting information such as log information. In the medical context, the issue description 106 may include a natural language description of signs and symptoms exhibited by the patient and may further include supporting test information that indicates the patient's health state.

Before an LLM 104 is used to determine the cause of the issue and suggest a solution, graph retrieval augmented generation (RAG) 108 is used to identify the most relevant material 110 from the source information 102. The graph RAG 108 determines a similarity score between the issue description 106 and the different elements of the source information 102. The most relevant documents are selected as context for the LLM 104 when prompted with the issue description 106.

The LLM 104 then generates a solution 112 to the issue. In software development embodiments, the solution 112 may include a patch to the source information 102, for example changing the source code of the software project to fix a bug. In medical embodiments, the solution 112 may include a treatment recommendation that diagnoses the patient and identifies treatments that will help with the issue. In some cases, a treatment recommendation may trigger an automatic treatment action.

Finding the relevant documents can be particularly helpful when working with large volumes of source information 102. In some case, the only way to find files may be by their file names, which often are not descriptive of their contents. Assessing the similarity of these documents to the issue description 106 makes it possible to determine when the contents will be pertinent to solving an issue.

In some embodiments, a similarity score may be determined by comparing the issue description 106 to each document in the source information 102. This process may include encoding the issue description 106 as a vector in a latent space and applying a similar encoding to each of the documents in the source information 102. This encoding may be performed using any appropriate natural language encoder, where documents that have similar contents will have vectors that point to similar locations within the latent space. In some embodiments the similarity score may be determined using the cosine similarity between two vectors. In some embodiments the similarity may be determined using other similarity metrics, such as BertScore or recall-oriented understudy for gisting evaluation (ROUGE).

Identifying relationships encourages a better understanding of the entire co0de base. The relationships involved may include import relationships (e.g., a #include instruction in a source code file). For example, importing Pyton will use other file definitions for functions. The relationship graph may therefore denote the file relationships identified by such import statements. The relationship helps to include related files to read into the context for the LLM.

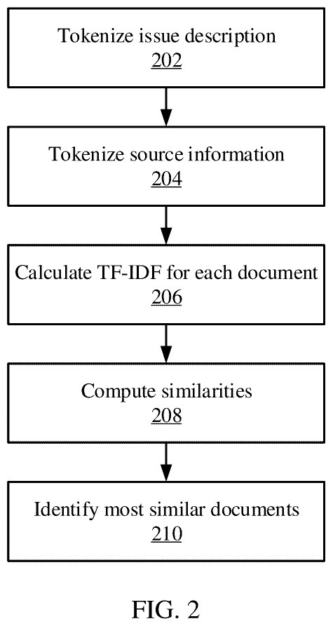

Referring now to FIG. 2, a method for identifying the most relevant material 110 is shown. Block 202 tokenizes the issue description 106 and block 204 tokenizes the documents of the source information 102. These tokens may be mapped to word identifier numbers, so that each word may be assigned a unique number value.

Block 206 calculates a TF-IDF (term frequency, inverse document frequency) value for each token to evaluate the importance of that token in each of the documents. This may include determining a term frequency for a given token t and a given document d as the number of times the token t appears in document d, divided by the number of terms in that document. An inverse document frequency for the token t can be determined as the log of a total number of documents, divided by the number of documents containing the token t. The TF-IDF for the token t and the document d is then calculated as the product of the term frequency and the inverse document frequency:

TF−IDF=TF(t,d)×IDF(t)

Block 208 computes similarities, for example using the cosine similarity metric and the similarity matrix. The similarity matrix may be built by calculating the TF-IDF matrix where each document is a row of the matrix and each column is a word. The values of the matrix may be the term frequency, multiplied by the inverse document frequency. Block 210 selects the top k most similar documents based on their similarity scores, for example using a priority queue. The cosine similarity may be used to search relevant files to be included in the LLM context.

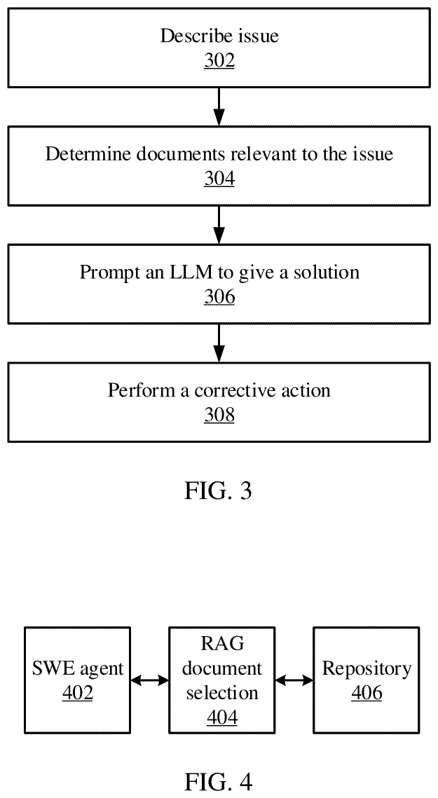

Referring now to FIG. 3, a method of using RAG to correct an issue is shown. Block 302 creates issue description 106 based on some indication of a problem with a system. In software embodiments, the issue description 106 may include a natural language description of the issue, including the circumstances that caused the issue and any effects that it had. The issue description 106 may additionally include error messages and logs associated with the issue. In medical embodiments, the issue description 106 may include a natural language description of a medical condition of a patient, including signs and symptoms exhibited by the patient.

Block 304 identifies documents that are relevant to the issue, for example as described above in FIG. 2. In some embodiments, a predetermined number k of documents may be selected. In some embodiments, a number of documents may be adaptively selected based on the limitations of the LLM 104. For example, a most relevant documents may be selected according to a rank determined by the documents' similarity scores. Additional documents may be selected until the total number of tokens from the selected documents and the issue description would exceed a token limit of the LLM 104. This can help to maximize the amount of contextual information within the limits of the LLM system.

Block 306 then prompts the LLM 104 to give a solution 112, using the issue description 106 and the relevant material 110. In software embodiments, the solution 112 may include a patch or other recommendation for changes to make to the software project to correct the problem. In medical embodiments, the solution 112 may include treatment recommendations.

Block 308 performs a corrective action based on the solution 112. In software embodiments, the corrective action may include patching the source code of the software project, making some change to the build environment for the software project, or performing some other action that affects how the software is implemented and executed. In medical embodiments, the corrective action may include automatically performing the recommendation indicated in the solution 112, for example by administering a drug or halting the administration of some other treatment.

Referring now to FIG. 4, a software engineering (SWE) system with RAG-based code assistance is shown. A SWE agent 402 may include an LLM that generates code at the prompting of a software engineer. The SWE agent 402 may be given a prompt that seeks a solution to an issue, such as a bug in a software project. Rather than providing all of the documents in a cloned repository 406 as context to the prompt, RAG document selection 404 selects a set of the most relevant documents based on similarity to a description of the issue. Using the selected documents, the SWE agent 402 can then prompt the LLM to generate a solution to the issue and can implement that solution, for example by altering files stored in the repository 406.

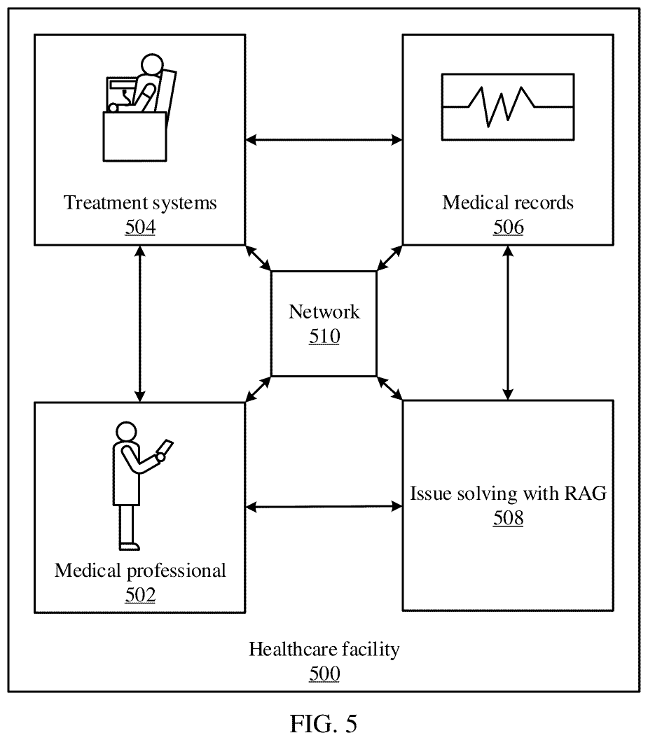

Referring now to FIG. 5, a diagram of RAG-based solutions to health issues is shown in the context of a healthcare facility 500. Issue solving with RAG 508 may be used to identify a treatment responsive to a patient's health condition, for example based on the patient's medical records 506 and general information relating to medical conditions. The healthcare facility may include one or more medical professionals 502 who review information extracted from a patient's medical records 506 to determine their healthcare and treatment needs. These medical records 506 may include self-reported information from the patient, test results, and notes by healthcare personnel made to the patient's file. Treatment systems 504 may furthermore monitor patient status to generate medical records 506 and may be designed to automatically administer and adjust treatments as needed.

Based on information drawn from the issue solving with RAG 508, the medical professionals 502 may then make medical decisions about patient healthcare suited to the patient's needs. For example, the medical professionals 502 may make treatment decisions based on a diagnosis generated by the issue solving with RAG 508 and may prescribe particular medications, surgeries, and/or therapies that are appropriate to the diagnosis disease.

The different elements of the healthcare facility 500 may communicate with one another via a network 510, for example using any appropriate wired or wireless communications protocol and medium. Thus issue solving with RAG 508 receives data from treatment systems 504, medical professionals 502, and from medical records 506, and searches the medical records 506 to identify documents that are most relevant to the patient's condition. The issue solving with RAG 508 may further coordinate with treatment systems 504 in some cases to automatically administer or alter a treatment. For example, if the solution indicates a particular treatment, the system may automatically trigger implement the treatment, such as by initiating or halting the administration of a medication.

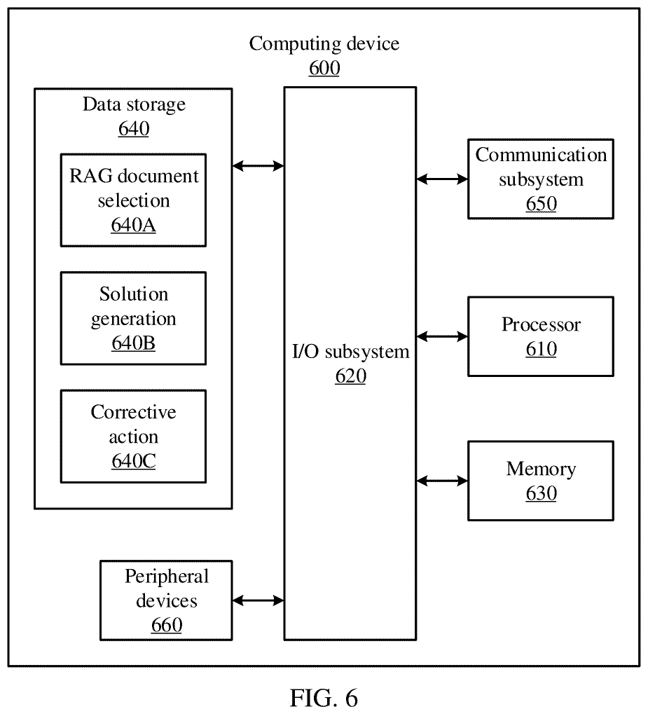

Referring now to FIG. 6, an exemplary computing device 600 is shown, in accordance with an embodiment of the present invention. The computing device 600 is configured to perform visual question answering.

The computing device 600 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 600 may be embodied as one or more compute sleds, memory sleds, or other racks, sleds, computing chassis, or other components of a physically disaggregated computing device.

As shown in FIG. 6, the computing device 600 illustratively includes the processor 610, an input/output subsystem 620, a memory 630, a data storage device 640, and a communication subsystem 650, and/or other components and devices commonly found in a server or similar computing device. The computing device 600 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 630, or portions thereof, may be incorporated in the processor 610 in some embodiments.

The processor 610 may be embodied as any type of processor capable of performing the functions described herein. The processor 610 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 630 may be embodied as any type of volatile or non-volatile memory or data storage capable of performing the functions described herein. In operation, the memory 630 may store various data and software used during operation of the computing device 600, such as operating systems, applications, programs, libraries, and drivers. The memory 630 is communicatively coupled to the processor 610 via the I/O subsystem 620, which may be embodied as circuitry and/or components to facilitate input/output operations with the processor 610, the memory 630, and other components of the computing device 600. For example, the I/O subsystem 620 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 620 may form a portion of a system-on-a-chip (SOC) and be incorporated, along with the processor 610, the memory 630, and other components of the computing device 600, on a single integrated circuit chip.

The data storage device 640 may be embodied as any type of device or devices configured for short-term or long-term storage of data such as, for example, memory devices and circuits, memory cards, hard disk drives, solid state drives, or other data storage devices. The data storage device 640 can store program code 640A for RAG document selection, 640B for generating solutions, and/or 640C for performing corrective actions. Any or all of these program code blocks may be included in a given computing system. The communication subsystem 650 of the computing device 600 may be embodied as any network interface controller or other communication circuit, device, or collection thereof, capable of enabling communications between the computing device 600 and other remote devices over a network. The communication subsystem 650 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 600 may also include one or more peripheral devices 660. The peripheral devices 660 may include any number of additional input/output devices, interface devices, and/or other peripheral devices. For example, in some embodiments, the peripheral devices 660 may include a display, touch screen, graphics circuitry, keyboard, mouse, speaker system, microphone, network interface, and/or other input/output devices, interface devices, and/or peripheral devices.

Of course, the computing device 600 may also include other elements (not shown), as readily contemplated by one of skill in the art, as well as omit certain elements. For example, various other sensors, input devices, and/or output devices can be included in computing device 600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 600 are readily contemplated by one of ordinary skill in the art given the teachings of the present invention provided herein.

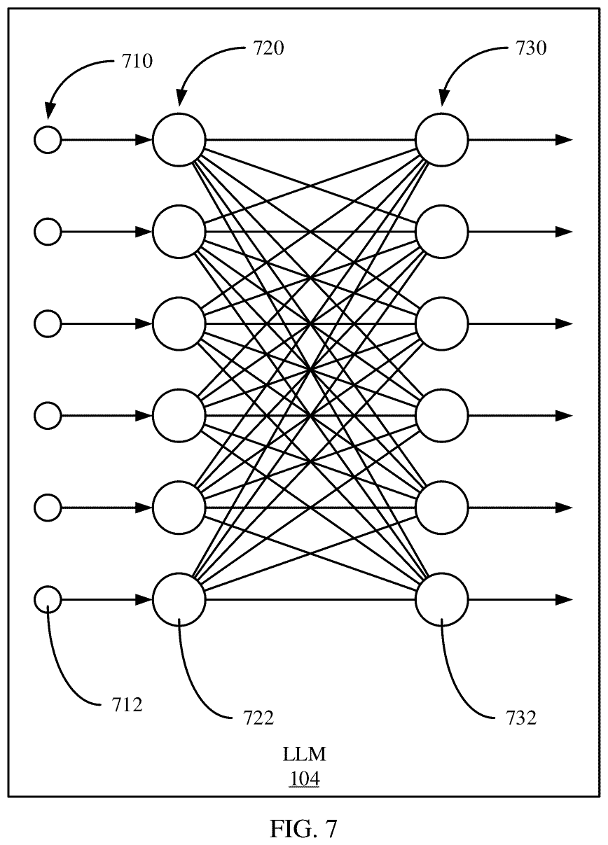

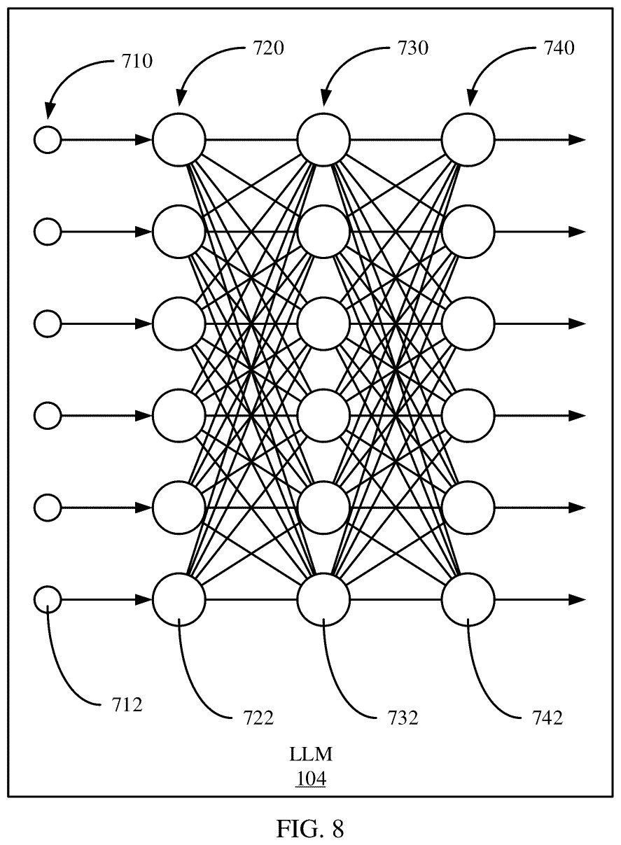

Referring now to FIGS. 7 and 8, exemplary neural network architectures are shown, which may be used to implement parts of the present machine learning models, such as the language model 104. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 720 of source nodes 722, and a single computation layer 730 having one or more computation nodes 732 that also act as output nodes, where there is a single computation node 732 for each possible category into which the input example could be classified. An input layer 720 can have a number of source nodes 722 equal to the number of data values 712 in the input data 710. The data values 712 in the input data 710 can be represented as a column vector. Each computation node 732 in the computation layer 730 generates a linear combination of weighted values from the input data 710 fed into input nodes 720, and applies a non-linear activation function that is differentiable to the sum. The exemplary simple neural network can perform classification on linearly separable examples (e.g., patterns).

A deep neural network, such as a multilayer perceptron, can have an input layer 720 of source nodes 722, one or more computation layer(s) 730 having one or more computation nodes 732, and an output layer 740, where there is a single output node 742 for each possible category into which the input example could be classified. An input layer 720 can have a number of source nodes 722 equal to the number of data values 712 in the input data 710. The computation nodes 732 in the computation layer(s) 730 can also be referred to as hidden layers, because they are between the source nodes 722 and output node(s) 742 and are not directly observed. Each node 732, 742 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 732 in the one or more computation (hidden) layer(s) 730 perform a nonlinear transformation on the input data 712 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
comparing a description of an issue to a plurality of documents to generate similarity scores for the plurality of documents;
selecting a set of most-relevant documents from the plurality of documents based on the similarity scores;
prompting a large language model (LLM) to generate a solution to the issue; and
performing a corrective action based on the solution to correct the issue.

2. The method of claim 1, wherein comparing the description of the issue to the plurality of documents includes comparing vector representations of the description and the plurality of documents using a similarity metric to generate the similarity scores.

3. The method of claim 1, wherein selecting the set of most-relevant documents includes selecting a number of documents in accordance with a limitation of the LLM.

4. The method of claim 3, wherein the set of most-relevant documents includes a maximum number of documents having highest similarity scores of the plurality of documents without exceeding a token limit of the LLM.

5. The method of claim 1, wherein the issue description identifies a bug in a software project and wherein the corrective action includes patching a file in the software project to fix the bug.

6. The method of claim 1, wherein the issue description identifies a health condition of a patient and wherein the corrective action includes automatically administering a treatment to the patient to treat the health condition.

7. The method of claim 6, wherein the plurality of documents include medical records of the patient.

8. The method of claim 6, wherein the solution is used for medical decision making.

9. The method of claim 1, wherein comparing the description of the issue to the plurality of documents includes computing a TF-IDF (term frequency, inverse document frequency) for the plurality of documents.

10. The method of claim 1, wherein the large language model is a trained machine learning model that accepts the set of most-relevant documents as context to a prompt to generate the solution.

11. A system, comprising:
a hardware processor; and
a memory that stores a computer program which, when executed by the hardware processor, causes the hardware processor to:
compare a description of an issue to a plurality of documents to generate similarity scores for the plurality of documents;
select a set of most-relevant documents from the plurality of documents based on the similarity scores;
prompt a large language model (LLM) to generate a solution to the issue; and
perform a corrective action based on the solution to correct the issue.

12. The system of claim 11, wherein the comparison of the description of the issue to the plurality of documents includes a comparison of vector representations of the description and the plurality of documents using a similarity metric to generate the similarity scores.

13. The system of claim 11, wherein selection of the set of most-relevant documents includes selection of a number of documents in accordance with a limitation of the LLM.

14. The system of claim 13, wherein the set of most-relevant documents includes a maximum number of documents having highest similarity scores of the plurality of documents without exceeding a token limit of the LLM.

15. The system of claim 11, wherein the issue description identifies a bug in a software project and wherein the corrective action includes patching a file in the software project to fix the bug.

16. The system of claim 11, wherein the issue description identifies a health condition of a patient and wherein the corrective action includes automatically administering a treatment to the patient to treat the health condition.

17. The system of claim 16, wherein the plurality of documents include medical records of the patient.

18. The system of claim 16, wherein the solution is used for medical decision making.

19. The system of claim 11, wherein the comparison of the description of the issue to the plurality of documents includes computing a TF-IDF (term frequency, inverse document frequency) for the plurality of documents.

20. The system of claim 11, wherein the large language model is a trained machine learning model that accepts the set of most-relevant documents as context to a prompt to generate the solution.
